(19) United States
(12) Patent Application Publication
(10) Pub. No.: US 2023/0374901 A1
(43) Pub. Date: Nov. 23, 2023

(54) METHOD AND SYSTEMS FOR REAL-TIME MEASUREMENT OF REID VAPOR PRESSURE IN FLUIDS

(71) Applicant: FMC Technologies, Inc., Houston, TX (US)

(72) Inventors: Sander Baaren, Houston, TX (US); Ryan Malone, Houston, TX (US); Eric Rasmussen, Houston, TX (US); Victor Van Asperen, Houston, TX (US)

(73) Assignee: FMC Technologies, Inc., Houston, TX (US)

(21) Appl. No.: 18/247,215

(22) PCT Filed: Nov. 5, 2021
(86) PCT No.: PCT/US2021/058168

§ 371 Date: Mar. 29, 2023

Related U.S. Application Data
(60) Provisional application No. 63/113,661, filed on Nov. 13, 2020.

(51) Int. Cl. E21B 47/06; E21B 43/12

(52) U.S. Cl. CPC E21B 47/06; E21B 43/121; E21B 2200/20

(57) ABSTRACT

A vapor pressure monitoring system may include a plurality of sensors disposed on equipment at a processing facility during one or more stages of refining and/or processing fluids. The plurality of sensors may be configured to monitor one or more properties of the fluid. In addition, one or more transmitters may be configured to transmit the one or more properties from the plurality of sensors to a computer system. The computer system may be configured to determine vapor pressure of the fluids based on the one or more fluid properties.

### BACKGROUND

Fluids are typically produced from a reservoir in a formation by drilling a wellbore into the formation, establishing a flow path between the reservoir and the wellbore, and conveying the fluids from the reservoir to the surface through the wellbore. Fluids produced from a hydrocarbon reservoir may include natural gas, oil, and water. Typically, once the fluids are at the surface, the fluids may then be transported to be refined and/or processed at a processing facility or a production facility for distribution. The fluids may be refined and/or processed to be various products such as gasoline or petrol, kerosene, jet fuel, diesel oil, heating oil, fuel oils, lubricants, waxes, asphalt, natural gas, and liquefied petroleum gas (LPG) as well as hundreds of petrochemicals. Typically, various fluid characteristics may be measured by taking samples from the fluids to ensure the fluids meet government regulations (e.g., Environmental Protection Agency (“EPA”)) and other requirements (e.g., customer standards).

As shown in FIG. 1, one or more wells 1, 2 may produce fluids, such as oil, gas, and water, to send to a processing facility 3 or a production facility via pipelines and tankers. The one or more wells 1, 2 may be land based and/or offshore based. Additionally, the processing facility 3 may also receive fluids from various other sources 4 such as a fluid storage or other production facilities. The processing facility 3 may be a facility having equipment to refine and/or process the fluids to make the fluids ready for sale and to meet government regulations or customer standards (e.g., various fluid properties such as fluid composition and allowable impurities). From the processing facility 3, the refined and/or processed fluids may be distributed to various customers (5, 6, 7, 8, 9). For example, the various customers may be a household 5, office buildings 6, manufacturers and gas stations 7, power plants 8, and storage tanks 9.

In assessing the fluids being refined and/or processed at the processing facility 3, knowing a vapor pressure of the fluids may play a critical role in the production of the fluids. The vapor pressure is the pressure at which a liquid and vapor of the fluids are in equilibrium at a given temperature. In other words, the vapor pressure is a measure of the tendency of fluid to change into the gaseous or vapor state and increases with temperature. The temperature at which the vapor pressure at the surface of a liquid becomes equal to the pressure exerted by the surroundings is called the boiling point of the liquid. Typically, government regulations and customer standards may dictate a limit of vapor pressure allowed for the fluids being distributed from the processing facility 3. Therefore, at the processing facility 3, measuring and determining the vapor pressure of the fluids play an important role in producing the fluids for distribution.

### SUMMARY

This summary is provided to introduce a selection of concepts that are further described below in the detailed description. This summary is not intended to identify key or essential features of the claimed subject matter, nor is it intended to be used as an aid in limiting the scope of the claimed subject matter.

In one aspect, embodiments disclosed herein relate to a vapor pressure monitoring system. The vapor pressure monitoring system may include a plurality of sensors disposed on equipment at a processing facility during one or more stages of refining and/or processing fluids. The plurality of sensors may be configured to monitor one or more properties of the fluid. In addition, one or more transmitters may be configured to transmit the one or more properties from the plurality of sensors to a computer system. The computer system may be configured to determine vapor pressure of the fluids based on the one or more fluid properties.

In another aspect, embodiments disclosed herein relate to a method. The method may include monitoring one or more fluid properties of a fluid with a plurality of sensors at one or more stages in a processing facility. The method may also include transmitting the one or more fluid properties, via one or more transmitters, to a computer system. The method may further include determining, with the computer system, vapor pressure of the fluid based on the one or more fluid properties.

In yet another aspect, embodiments disclosed herein relate to a non-transitory computer readable medium storing instructions on a memory coupled to a processor. The instructions may include functionality for obtaining one or more fluid properties of a fluid at one or more stages in a processing facility. The processor may be configured to determine vapor pressure of the fluid based on the one or more fluid properties and display the determined vapor pressure on a display coupled to the processor.

Other aspects and advantages of the invention will be apparent from the following description and the appended claims.

### BRIEF DESCRIPTION OF DRAWINGS

The following is a description of the figures in the accompanying drawings. In the drawings, identical reference numbers identify similar elements or acts. The sizes and relative positions of elements in the drawings are not necessarily drawn to scale. For example, the shapes of various elements and angles are not necessarily drawn to scale, and some of these elements may be arbitrarily enlarged and positioned to improve drawing legibility. Further, shapes of the elements as drawn are not necessarily intended to convey any information regarding the actual shape of the elements and have been solely selected for ease of recognition in the drawing.

FIG. 1 is a schematic diagram of producing fluids in accordance with prior art.

FIG. 2 is a schematic diagram of a system for monitoring fluids at a processing facility in accordance with one or more embodiments disclosed herein.

FIG. 3 is a schematic diagram of a vapor pressure monitoring system at a processing facility in accordance with one or more embodiments disclosed herein.

FIGS. 4A-4C are schematic diagrams of a control system of a vapor pressure monitoring system in accordance with one or more embodiments disclosed herein

FIGS. 5 and 6 are flow charts of a method in accordance with one or more embodiments disclosed herein.

FIG. 7 is a schematic diagram of a computing system in accordance with embodiments disclosed herein.

### DETAILED DESCRIPTION

In the following detailed description, certain specific details are set forth to provide a thorough understanding of various disclosed implementations and embodiments. However, one skilled in the relevant art will recognize that implementations and embodiments may be practiced without one or more of these specific details, or with other methods, components, materials, and so forth. For the sake of continuity, and in the interest of conciseness, same or similar reference characters may be used for same or similar objects in multiple figures. As used herein, the term “coupled” or “coupled to” or “connected” or “connected to” “attached” or “attached to” may indicate establishing either a direct or indirect connection, and is not limited to either unless expressly referenced as such. As used herein, fluids may refer to slurries, liquids, gases, and/or mixtures thereof.

Typically, in the oil and gas industry, vapor pressure of a fluid may be referred to as a measurement of a volatility of the fluids, such as gasoline, crude oil, or other petroleum products. The vapor pressure is the property of the fluid based on evaporation characteristics. For example, the vapor pressure may be defined as vapor pressure exerted by vapor of the fluids. The vapor pressure is commonly reported in kilopascals (“kPa”) or pounds per square inch (“psi”). Typically, when the vapor pressure is a Reid vapor pressure (“RVP”), the RVP may be determined according to ISO 3007:1999 or American Society for Testing Materials (“ASTM”) Standard D323. According to the ASTM Standard D323, a sample of the fluid is placed in a container such that the ratio of the vapor volume to the liquid volume is 4 to 1 and the absolute pressure at 100° F. (37.8° C.) in the container is the RVP for the fluid. In other words, the RVP is the vapor pressure at 100° F. of the fluid determined in a volume of vapor four times the liquid volume.

Since a portion of the fluid has been vaporized to fill the vapor space, the fluid has lost some of the lighter components. This effectively changes the composition of the fluid and thus yields a slightly lower vapor pressure than the true vapor pressure of the fluid in its original composition. The RVP of the fluid, then, is slightly lower than the true vapor pressure (“TVP”) of the fluid at 100° F. (37.8° C.). The TVP may be defined as an equilibrium partial pressure exerted by a volatile organic liquid as a function of temperature as determined by the test method ASTM D2879. The RVP may differ from the TVP of the fluids. More specifically, the RVP is the vapor pressure measured at 37.8° C. (100° F.) and the TVP is a function of the temperature. Additionally, the RVP is defined as being measured at a vapor-to-liquid ratio of 4:1, whereas the TVP of the fluids may depend on the actual vapor-to-liquid ratio. In addition, the RVP may include the pressure associated with the presence of dissolved water and air in a sample of the fluid, which is excluded by some but not all definitions of the TVP. Further, testing for the RVP is applied to a sample which has had the opportunity to volatize somewhat prior to measurement. For example, the sample container may be required to be only 70-80% full of liquid so that whatever volatizes into the container headspace is lost prior to analysis. The sample then again volatizes into the headspace of the D323 test chamber before it is heated to 37.8 degrees Celsius.

Despite the inaccuracy of RVP measurements, the RVP is used to specify volatility limits for gasoline, crude oil, or other petroleum products in sales contracts. Stabilization systems may be designed to meet RVP requirements because the RVP of a fluid is always less than the true vapor pressure at 100° F. (37.8° C.). Therefore, the separation in the stabilizer should be designed to yield a mixture with the TVP at 100° F. (37.8° C.) equal to the RVP requirement. This will yield a product with the RVP slightly below the required RVP.

The RVP is important relating to the function and operation of gasoline, crude oil, or other petroleum powered products. For example, a higher RVP for more vaporization may be desirable during cold months while a lower RVP for less vaporization may be desirable during warm months. Additionally, the RVP may be specified by customers, such as crude oil purchasers. In another example, if the crude oil is to be transported by tanker or truck prior to reaching a processing plant, customers may specify a low RVP so that they will not be paying for light components in the fluid, which will be lost due to weathering. Vapor losses in the crude oil may not only cause measurable financial impact from a sales perspective but could lead to elevated risk from a hazards perspective (e.g., fires, explosions, or poisonous gases). For crude oil, regulator committees, such as the Occupational Safety and Health Administration (“OSHA”) may set exposure limits on some of the gaseous components (e.g., H2S, benzene, etc.) that vaporize from crude oils. For gasoline and fuel, the RVP is important for both performance and environmental reasons. First, because engines require the fuel to be vaporized in order to burn, gasoline must meet a minimum RVP to ensure that it is volatile enough to vaporize under cold start conditions. Engines also have a maximum limit for RVP set by concerns over vaporization in the fuel line that can result in vapor lock, or a blocking of the fuel line. However, the most critical limit RVP in most markets now is environmental concern about evaporative emissions outside of the vehicle, which contribute to pollution. Typically, it is this concern that sets the critical maximum RVP specification for most grades of gasoline. Additionally, government entities, such as the Environmental Protection Agency (“EPA”), may regulate the RVP of gasoline, crude oil, or other petroleum products sold during the summer ozone season (June 1 to September 15) to reduce evaporative emissions from gasoline that contribute to ground-level ozone and diminish the effects of ozone-related health problems. Typically, the RVP may range from 7 psi to 15 psi or 48 kPa to 103 kPa. Based on government regulations and customer standards, production facilities or refineries manipulate the RVP of the fluid to maintain a fluid reliability.

Embodiments disclosed herein are directed to methods and systems to real-time monitoring and determining a vapor pressure of fluids at a processing facility. More specifically, embodiments disclosed herein are directed to the real-time monitoring of the vapor pressure based on establishing the temperature and pressure at various steps in the processing facility to refine the fluids, to form gasoline, crude oil, or other petroleum products, such that a certain amount of volatile components have been removed or are still present which inherently may be converted into an vapor pressure value. The vapor pressure may include a Reid vapor pressure (RVP) or a true vapor pressure (TVP). The different embodiments described herein may provide methods and systems for real-time measurement of the vapor pressure of the fluids that plays a valuable and useful role in producing fluids at the processing facility. By using the methods and systems for real-time measurement of the vapor pressure of the fluids at the processing facility, the vapor pressure may be monitored and adjusted to meet government regulations and customer standards to avoid nonproductive down-time (NPT) and costly fluid treatment. Further, a configuration and arrangement of monitoring equipment at the processing facility to the monitor and adjust the vapor pressure according to one or more embodiments described herein may provide a cost-effective alternative to conventional methods used at facilities in vapor pressure analyzation. For example, one or more embodiments described herein may eliminate the need for a specialized operator and other costly testing equipment at the facilities conventionally used in vapor pressure analyzation. The embodiments are described merely as examples of useful applications, which are not limited to any specific details of the embodiments herein.

In accordance with one or more embodiments, a vapor pressure monitoring system includes positioning a plurality of sensors on equipment at a processing facility during various stages of refining and/or processing fluids, such as gasoline, crude oil, or other petroleum products. The plurality of sensors may monitor various fluid properties of the fluid. In one or more embodiments, one or more transmitters may transmit data from the plurality of sensors to a control system. The control system may be a computer system having a memory coupled to a processor. In some embodiments, the control system may be replaced with a computer or data system without control functionality. The control system may then use the data received from the one or more transmitters to determine the vapor pressure of the fluids. In addition, the control system may display the vapor pressure of the fluids on a display for a user to access. Further, the control system may send alerts to a user and/or automatically adjust the equipment at the processing facility to change various fluid properties to ensure the vapor pressure of the fluids meets government regulations and customer standards.

Conventional methods in the oil and gas industry typically requires manually operated vapor pressure analyzers using fluid samples to determine the vapor pressure. Conventionally, an operator needs to travel to a certain facility location, take a sample, run it through the vapor pressure analyzer and clean up afterwards. For fluids such as gasoline, crude oil, or other petroleum products, cleaning is essential and must be done, at a minimum, after every 5-10 samples taken. In some conventional methods, automated vapor pressure analyzers automatically draw the fluid sample from a pipeline before the fluid is sold. The automated vapor pressure analyzers are a batch process where analyzing a fluid sample takes around 5-15 minutes. However, a major drawback for automated vapor pressure analyzers is that for crude oil on a processing facility, the automated vapor pressure analyzers also need cleaning and maintenance after every 5-10 samples. Consequently, the number of samples per day that may practically be analyzed is very limited for unmanned facilities that are often in remote locations. Because of the limitations on manually or automated vapor pressure analyzers, the equipment is large and costly and is limited to only certain stages of the facilities. Additionally, there may be many hours or days of processing where the fluid may not meet the vapor pressure requirement. This can lead to issues when the oil is trucked or is temporarily stored in tanks increasing NPT and cost. In addition, the customer of the fluid may check if the fluid meets the vapor pressure requirement, where certain contracts allow the customer to refuse taking more fluid or impose penalties if the vapor pressure requirement of the fluid is not met. In addition, customers may unnecessarily increase energy input into the fluid or increase storage times to ensure vapor pressure requirements are met, and this unnecessarily increases operating costs greenhouse gas emissions, and reduces oil production efficiency or yield.

Advantageously, the vapor pressure monitoring system disclosed herein may provide real-time vapor pressure measurement of a fluid at any stage in a processing facility without costly testing equipment and operators used in typical vapor pressure analyzation methods. Moreover, because the vapor pressure of the fluid is measured at any stage in the processing facility, processing equipment may be adjusted in real-time to maintain and ensure the vapor pressure requirement of fluid is met, without costly NPT. Overall, the vapor pressure monitoring systems herein may minimize product engineering, risk associated with operators at processing facilities, and may provide reduction of assembly time and NPT, a hardware cost reduction, and weight and envelope reduction. Thus, the disclosed vapor pressure monitoring of fluids using the vapor pressure monitoring systems herein improves safety on site and reduces cost associated with conventional vapor pressure analyzation operations.

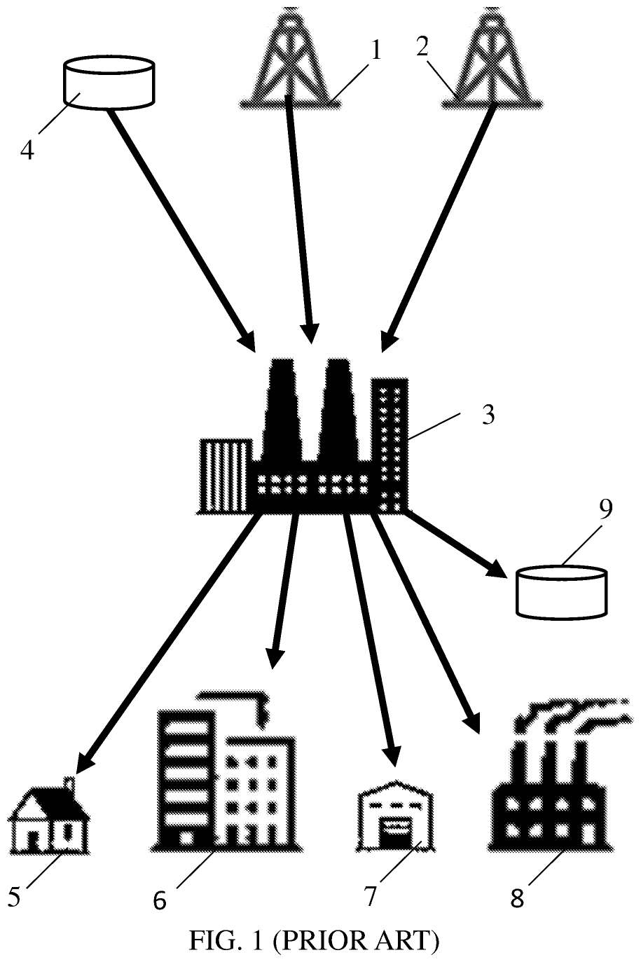

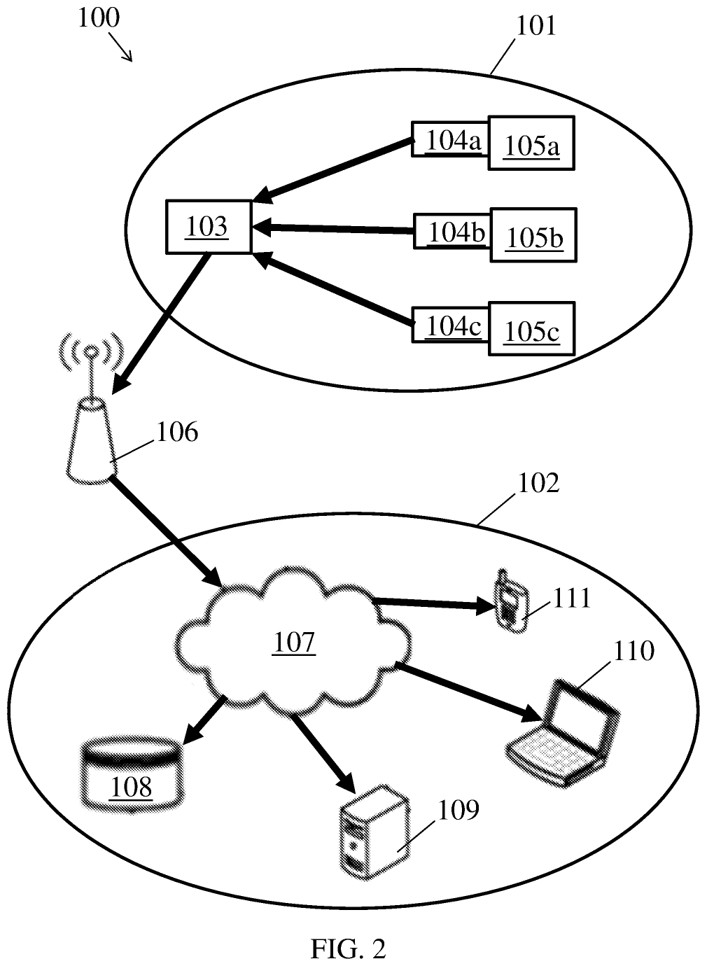

Referring to FIG. 2, a vapor pressure monitoring system 100 in accordance with embodiments disclosed herein is illustrated. The vapor pressure monitoring system 100 may include one or more sensor networks 101 in communication, either wirelessly or wired, with a server network 102. More specifically, the one or more sensor networks 101 may include one or more transmitters 103 and a plurality of sensors 104a-104c. In one or more embodiments, the plurality of sensors 104a-104c are disposed near, or attached to, equipment 105a-105c at a processing facility to be monitored. For example, the equipment 105a-105c may be any equipment which has fluids therein at the processing facility such as storage tanks, pipelines, compressors, separators, heater treaters, heat exchangers, condensers, reboilers, reactors, and other fluid treatment equipment and/or their associated flow lines. Accordingly, the plurality of sensors 104a-104c themselves may be of several different types, each sensor (104a-104c) being adapted for a particular purpose with respect to the fluid, e.g., temperature sensing, pressure sensing, mass or volume flow metering, proximity sensing, density sensing, viscosity sensing, environmental sensing, photo or video capturing, acceleration sensing, or any other type of sensor that may find applicability in the field. It is noted that while three sensors (104a-104c) and three pieces of equipment (105a-105c) are shown in FIG. 1, this is merely for example purposes only and any number of sensors and pieces of equipment may be used. Each sensor (104a-104c) may communicate, either wirelessly or wired, with the one or more transmitters 103. The one or more transmitters 103 may be located at a central site at the processing facility. It is further envisioned that the one or more transmitters 103 may be incorporated into each sensor (104a-104c). Furthermore, in accordance with one or more embodiments, the plurality of sensors 104a-104c may communicate between themselves, wirelessly or wired.

In addition, for the one or more transmitters 103 within range of any sensor (104a-104c), the one or more transmitters 103 may relay data from the plurality of sensors 104a-104c to the server network 102 by way of one or more base station 106, such as a cellular tower, connected to a wide area network (not shown). Each sensor (104a-104c) may not need to wirelessly communicate with the one or more transmitters 103, but may, in certain circumstances, communicate by wired connection. Further, the plurality of sensors 104a-104c and/or the one or more transmitters 103 may be equipped with a global positioning system (GPS) module including a GPS receiver and chipset configured to geolocate the equipment 105a-105c.

In some embodiments, the server network 102 may be configured to acquire and/or store data from the plurality of sensors 104a-104c in a memory located within the vapor pressure monitoring system 100. Once the plurality of sensors 104a-104c record data, the data is transmitted to the server network 102 via the one or more transmitters 103 and/or the base station 106. The one or more transmitters 103 and the base station 106 may encode and transmit this data in an appropriate form and under the appropriate set of protocols for transmission over a cellular network or a locally wired network. For example, any known wireless communication method may be used by the wireless gateway, e.g., GSM, CDMA, OFDMA, etc. One of ordinary skill will appreciate that cellular networks and locally wired networks are generally known in the art and, thus, for the sake of clarity and compactness, the details of the numerous known communication schemes will not be discussed in detail here. However, one of ordinary skill will appreciate that the wireless gateway may communicate by way of the cellular network under protocols defined within the various telecommunication standards, including but not limited to 3G, WiMAX, 4G-LTE, 5G, or other telecommunication standards.

One of ordinary skill will also appreciate that access to the cellular network infrastructure also integrates each sensor (104a-104c) with a larger Internet 107, such as a cloud. Accordingly, each sensor (104a-104c) may communicate through the cellular network-internet infrastructure to exchange data with the server network 102. In one or more embodiments, the server network 102 may include one or more remote data storage facilities 108, remote data server 109 that may itself include a local data storage facility, a computer 110, and mobile computing device 111, e.g., a cellular phone, smart phone, tablet PC, or handheld devices. As used herein, a data storage facility includes a cloud based remote data center, or any other system that includes network accessible memory locations. Accordingly, the data acquired by the plurality of sensors 104a-104c may be easily accessible anywhere where internet access or cellular service is available. One of ordinary skill will appreciate that the system may also be deployed within smaller scale local area networks (LANs) or wide area networks (WANs) without departing from the scope of the present disclosure. It is further envisioned that any component of the server network 102 may act as a control system for the vapor pressure monitoring system 100.

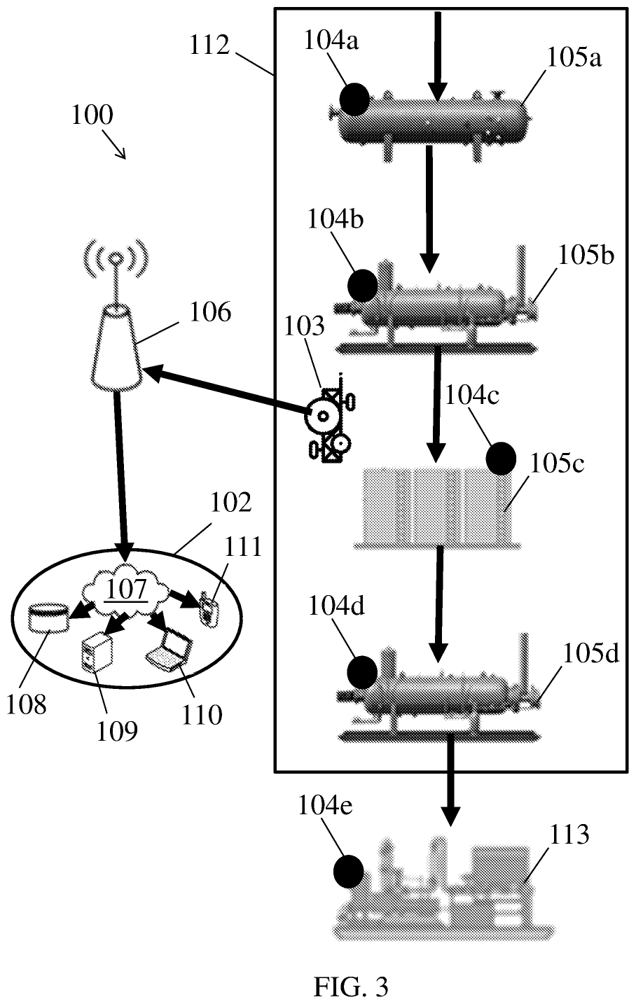

Referring to FIG. 3, the vapor pressure monitoring system 100 of FIG. 2 is illustrated at a processing facility 112 in accordance with embodiments disclosed herein. The processing facility 112 may include various equipment such as one or more initial separators 105a, one or more heater treaters 105b, one or more tanks 105c, one or more final separators 105d to refine and/or process fluids from a well. For example, the processing facility 112 may receive fluids such that the fluids enter the one or more initial separators 105a as a first stage. From the one or more initial separators 105a, the fluids may flow to the one or more heater treaters 105b as a second stage. From the one or more heater treaters 105b, the fluids may flow to the one or more tanks 105c as a third stage. From the one or more tanks 105c, the fluids may flow to the one or more final separators 105d as a fourth stage. It is noted that while only four stages are shown in FIG. 3, the processing facility 112 may include any number of stages to refine and/or process the fluids. Each stage may process or convert the fluids, withdrawing a portion of the fluids, such as a vapor product, and forwarding liquids to the next stage. Additionally, all four of the stages at the processing facility 112 may be operated at different temperatures and pressures. From the one or more final separators 105d, the refined and/or processed fluids may be distributed to an end customer facility 113 such as a manufacturer, a gas station, a power plant, or other facilities procuring the fluids.

In one or more embodiments, the plurality of sensors 104a-104d (as described in FIG. 2) may be disposed on or within the various equipment (105a-105d) within the processing facility 112. Additionally, the one or more transmitters 103 may be provided at a location within the processing facility 112. The location of the one or more transmitters 103 may be centralized to communicate with the plurality of sensors 104a-104d. The plurality of sensors 104a-104d may be used to collect data on various fluid properties of the fluid at the various stages (first-fourth) within the processing facility 112. For example, the collected data may include temperature readings, pressure readings, density readings, mass or volume flow readings, viscosity readings, or any other type of fluid properties. In some embodiments, the various equipment (105a-105d) may each have multiple sensors (104a-104d) such that each sensor of the multiple sensors (104a-104d) may be specific to one fluid property of the fluid. In a non-limiting example, each sensor of the plurality of sensors 104a-104d may have an antenna (not shown) to be in communication with the one or more transmitters 103. Alternatively, where both the network and the sensors are located at a common facility, the sensors may transmit data via wired connections to the network.

With the one or more transmitters 103, the collected data from the plurality of sensors 104a-104d may be sent to the server network 102 (as described in FIG. 2) at a location either away or at the processing facility 112. By obtaining the collected data, a control system of the server network 102 may then convert the collected data into various fluid property values to determine a vapor pressure of the fluids. In a non-limiting example, the control system may plot and cross-reference the collected data onto a three-dimensional lookup table. The three-dimensional lookup table may contain pre-determined values for a vapor pressure based on different fluid property values. The three-dimensional lookup table may be populated in different manners, such as simulation software that may predict a vapor pressure of a fluid based on a certain composition. In other cases, samples of the fluid may be taken and analyzed in lab to feed into a simulation model and then populate or update the three-dimensional lookup table. For example, as crudes vary in composition, an analysis of a crude oil feedstock may be provided to enhance the calculations of vapor pressure resulting from processing of the crude oil. Further, the collected data may auto populate the three-dimensional lookup table based on direct measurements of the vapor pressure of the fluids. In other non-limiting examples, the control system may include algorithms or equations, such as volume blending equations, to automatically calculate the vapor pressure of the fluid based on using the collected data in the algorithms or equations.

In some embodiments, the plurality of sensors 104a-104d may be flow meters, such as Coriolis meters, on vapor and liquid outlets of at a minimum the the one or more final separators 105d where the fluid is being stabilized, or on the vapor and liquid outlets of two or more of, or all of, the separation stages (e.g., the one or more initial separators 105a and the one or more final separators 105d). With the Coriolis meters, data on oil shrinkage (liquid volume in minus liquid volume out) of the fluid may be collected. For example, when the fluids enter a stabilization portion of processing facility 112, the fluids may have a certain mass flowrate. For example purposes only, the flow rate may be 100,000 lb/hr. The mass flowrate may include the volatile components that need to be removed to meet the vapor pressure requirements. When the volatile components (e.g., light components) are removed, the stabilized fluid may have a smaller mass flowrate such as 93,000 lb/hr. The amount of removed volatile components per mass balance would, in this example, amount to 7,000 lb/hr which exits through the vapor outlet(s). The 7,000 lb/hr exiting is the lighter components removed during processing, and the 93,000 lb/hr liquid product has a lower vapor pressure than the feed as a result. In a non-limiting example, if the fluid meets a vapor pressure requirement with a shrinkage of 7% based on mass flowrate, but the sensing systems herein observe that the shrinkage has reduced to only 5%, then not enough volatile components have been removed to meet the vapor pressure requirements and alerts may be sent to a user. This reduction in mass and also volume flow of the fluid due to stabilization is typically referred to as shrinkage, and is a metric that may be used to directly calculate vapor pressure or to complement/increase accuracy of estimating vapor pressure using other fluid properties such as temperature and pressure.

In one or more embodiments, the control system may display the vapor pressure to a user in communication with the server network 102. In a non-limiting example, a display of the computer 110 or the mobile computing device 111 may be used to display the collected data and the determined vapor pressure by feeding into a supervisory control and data acquisition (SCADA) system. Based on the determined vapor pressure, the control system may send alerts to a user that the vapor pressure may not meet government regulation and customer standards. Further, the control system may display and have commands on how to adjust operations to correct the vapor pressure. In a non-limiting example, if the vapor pressure needs to be reduced, the control system may send commands to either increase the temperature of operations on the fluids or reduce the pressure of operations on the fluids or a combination thereof. At higher temperatures and/or lower pressures, the volatile components of the fluids may no longer be able to stay dissolved in the fluids and, the volatile components come out of the fluid in vapor form and may be separated. Once these volatile components are separated, the vapor pressure of the remaining fluids may be lowered until a certain value is met. The control system may then repeat the process of determining the vapor pressure and send alerts once the certain value is met by the overall process or within the various stages in the processing facility 112. It is further envisioned that the vapor pressure monitoring system 100 may only be applied to one stage (first-fourth) at a time or concurrently at all the stages (first-fourth) of the processing facility 112.

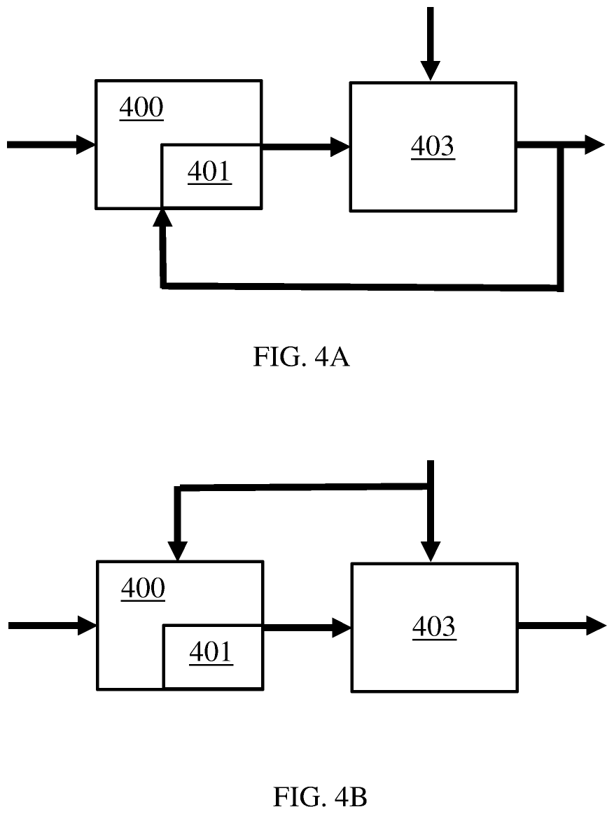

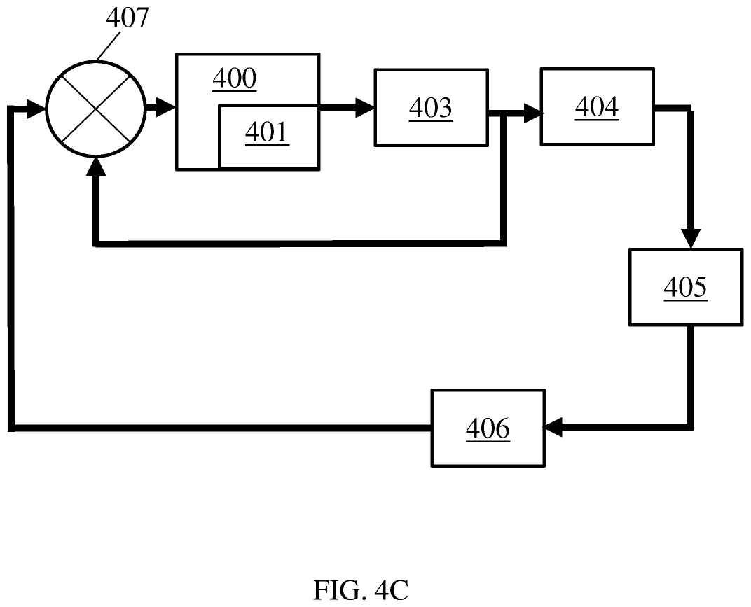

FIGS. 4A-4C, in one or more embodiments, illustrate various examples of a control system for the vapor pressure monitoring system 100. In FIGS. 4A and 4B, the control system 400 may include a controller 401 in a feedback control loop. The controller 401 may be steady state or dynamic type of controller. A user may enter vapor pressure setpoint in the control system 400 and the controller 401 may manipulate operations 403 to maintain the set vapor pressure. In a non-limiting example, the operations 403 may be fuel gas flow to a heater such that the controller 401 may control a fuel gas flow rate based on adjusting a heating medium temperature. In other words, the user sets the vapor pressure and the controller 401 may calculate and adjust process variables (fuel gas flow, heating medium temperature, heating medium by-pass) of the operations 403 to control the vapor pressure.

Referring to FIG. 4C, in one or more embodiments, the control system 400 may optimize the operations 403. After the operations 403, data reconciliation 404 may occur such that data may be collected. From the data reconciliation 404, parameter estimation models 405 may be run by the controller 401. The parameter estimation models 405 may be simulation models based on adjusting parameter in view of the data reconciliation 404. Once the parameter estimation models 405 are run, the controller 401 may be ready for optimization 406 and adjust process variables 407 of the operations 403 to control the vapor pressure. The optimization 406 may minimize operational cost (e.g., utility consumption) by determining key operational variables (e.g., pressures and temperatures throughout the operation) that will deliver a product fluid complying with vapor requirements whilst minimizing the operational cost. In a non-limiting example, the controller 401 may minimize gas consumption for heating (e.g., lowest separator pressure) whilst still ensuring the vapor pressure of the fluids meets government regulation and customer standards. It is further envisioned that when the vapor pressure requirement is difficult to achieve and an economic value is allocated to inlet and outlet streams of the operations 403, the optimization 406 by the controller 401 may increase the economics of the operations by maximizing the net economic result of the operations (revenue minus cost) by optimizing an oil yield from the fluid. More specifically, the optimization 406 by the controller 401 may determine the most economic mixture (e.g., oil/gas) of the fluid and gas production from the system given the market pricing of these petroleum products.

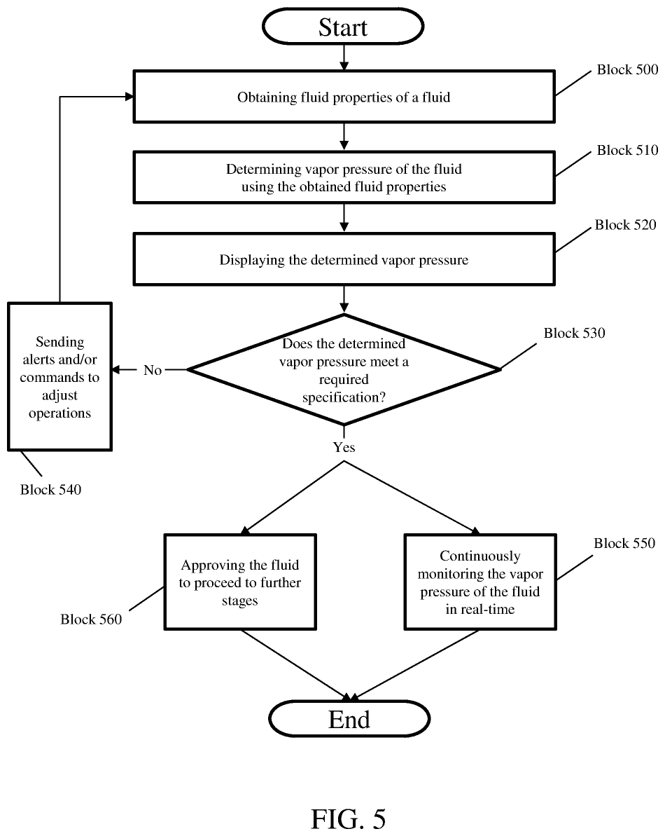

FIG. 5 is a flowchart showing a method of a monitoring a vapor pressure of a fluid at a processing facility using the vapor pressure monitoring system 100 of FIGS. 2-4C. One or more blocks in FIG. 5 may be performed by one or more components (e.g., the control system 400 coupled to the controller 401 in communication with the server network 102) as described in FIGS. 2-4C. For example, a non-transitory computer readable medium may store instructions on a memory coupled to a processor such that the instructions include functionality for operating the vapor pressure monitoring system 100. While the various blocks in FIG. 5 are presented and described sequentially, one of ordinary skill in the art will appreciate that some or all of the blocks may be executed in different orders, may be combined or omitted, and some or all of the blocks may be executed in parallel. Furthermore, the blocks may be performed actively or passively.

In Block 500, one or more measurements of fluid properties of a fluid are obtained in accordance with one or more embodiments. For example, a controller may obtain fluid property data from data packs in real-time taken from sensors coupled to equipment at a processing facility. In particular, one or more transmitters, in communication with the sensors, may transmit the data packs to the controller. Likewise, the fluid property data may correspond to various parameter fluid values, such as temperature, pressure, and fluid shrinkage readings. Moreover, the one or more transmitters and the sensors may form a network connection, such as an Ethernet connection, with the controller for transmitting fluid property data over a server network. In one embodiment, the fluid property data may correspond to a temperature and a pressure of the fluid in a separator. For example, a temperature and a pressure of the fluid may be obtained based on readings from sensors on or within the separator.

In Block 510, vapor pressure of the fluid is determined by using the one or more obtained fluid property measurements in accordance with one or more embodiments. For example, the controller uses one or more obtained fluid property measurements to calculate and determine the vapor pressure of the fluid. With the one or more obtained fluid property measurements, the controller is informed of the state of the fluid and the controller may cross-correlate and run simulation models on the fluid property measurements to determine the vapor pressure.

In Block 520, the determined vapor pressure may be displayed to a user in accordance with one or more embodiments. For example, the controller communicates with a device such as a computer or mobile computer to display the determined vapor pressure as well as the one or more obtained fluid property measurements.

In Block 530, a determination is made whether the determined vapor pressure meets a required specification in accordance with one or more embodiments. For example, the controller may obtain data over the server network identifying and determine the vapor pressure (see Blocks 500-520). If the answer to the determined vapor pressure meeting the required specification is no (e.g., the vapor pressure being too high or low), the controller may move to Block 540. In the Block 540, alerts and/or commands are sent to a user to request adjustments to the operations. For example, if the determined vapor pressure is higher than the required specification, the controller may send alerts to a user to either increase the temperature of the fluid or reduce the pressure of the fluid or a combination of the two to lower the vapor pressure. Further, the controller may automatically send commands to equipment to make the necessary adjustments to operations so the that the determine vapor pressure may change to meet required specification. Once alerts and commands are sent, the controller will go back to the Block 500 to repeat the previously mentioned Blocks (500-540) or until the determine vapor pressure meets the required specification. However, if the answer to the determined vapor pressure meeting the required specification is yes, the controller proceeds to Block 550 and/or Block 560.

In the Block 550, the vapor pressure of the fluid is continuously monitored in real-time. For example, the controller continuously repeats the Blocks 500-540 such that the vapor pressure of the fluid may be continuously determined during operations that may be continuous at the processing facility. In the Block 560, the fluid is approved to proceed to further stages in accordance with one or more embodiments. For example, the controller sends a command to the equipment to send the fluid to next stage for batch operations. Based on the stage that the vapor pressure is determined at, the controller may send alerts to a user on how to proceed. In one or more embodiments, the flowchart of FIG. 5 allows for the controller to continuously or batch monitor and determine the vapor pressure of the fluid in real-time at the processing facility. One skilled in the art will appreciate how utilizing the controller, the processing facility may produce a fluid with vapor pressure meeting the required specification.

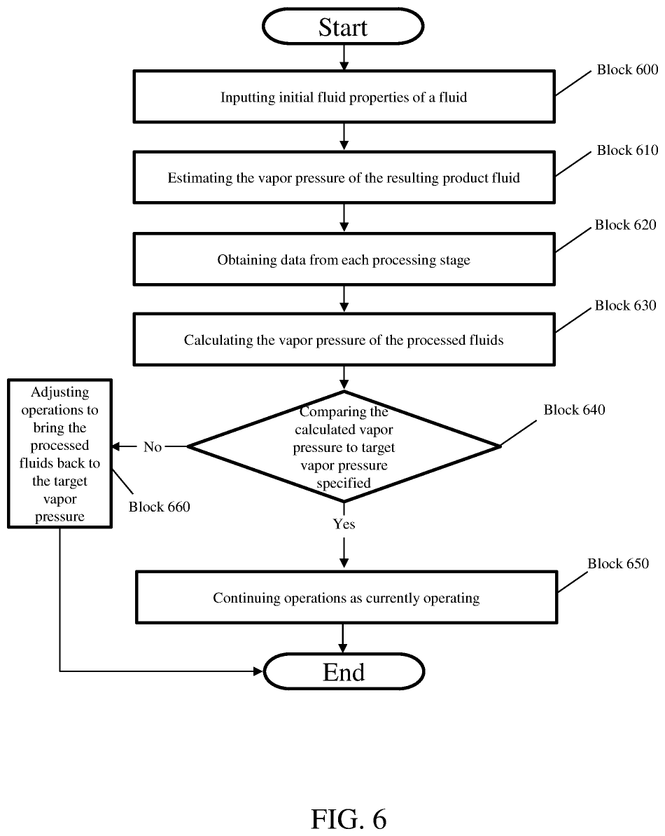

As another example, referring to FIG. 6, systems according to embodiments herein may be used to calculate a vapor pressure, such as a Reid or True Vapor Pressure, of a product based on the measured variables during processing of the fluid. In Block 500, initial fluid properties of the fluid may be input. For example, for a production facility receiving produced fluids from a well, the production facility may perform an initial separation of the crude oil from natural gases in the produced fluids. A “representative” initial composition may be input to the system, such as based on typical field compositions produced, measured compositions of the produced fluids, or an estimated composition based on the temperature and pressure of the formation from which the fluids are produced and/or the type of production being performed, such as primary or secondary oil recovery operations.

In Block 610, the vapor pressure of the resulting product fluid may be estimated. In some embodiments, as described above with respect to FIGS. 2-4, the vapor pressure of the final product may be estimated or calculated using a three-dimensional look up table based on the operations of, for example, the stabilizer (104d, 105). For example, the pressure and/or temperature of the stabilizer operations, and/or flow rates, may be used to calculate or estimate compositional changes in the fluid (changes from the initial feed to the first stage to the product output from the last stage), and thence to determine the vapor pressure of the product. From the initial composition, for example, the oil shrinkage (changes in feed to product flow rates) and/or the pressure and/or temperature of stabilizer operations may be used to estimate the proportion of lighter components removed from the composition, and to determine the vapor pressure based on the resulting composition.

To enhance the calculations during the Block 510, data may be obtained from each processing stage in Block 520. For example, temperature and/or pressure of separator 105a (FIG. 3) may be used to calculate a composition of fluids forwarded to heater treater 105b (FIG. 3). Further, sensors associated with heater treater 105b operations may be used to calculate a composition of the fluids forwarded to storage tanks 105c. Likewise, sensors associated with storage tanks 105c may be used to estimate a composition of the fluids forwarded to stabilizer 105d. Then, the sensors associated with the operations of stabilizer 105d may be used to estimate a composition of the fluids recovered as a product. From such data, the compositional changes may be estimated, and the vapor pressure of the product fluids may be calculated.

As described above, the compositional changes of the fluid from the initial to terminal processing steps, and thus the vapor pressure of the processed fluids recovered from the last processing stage, may be determined based on the conditions at one stage, such as the terminal stabilizing step, or multiple stages, such as the last two processing steps, the last three processing steps, all four processing steps, the first and last processing steps, or other combinations of two or more processing steps.

In this manner, the vapor pressure of the processed fluids may be calculated in Block 630. The calculated vapor pressure may be more accurate when data from multiple stages or each stage is included, as the compositional changes in the fluid may more accurately be reflected in the calculations. In Block 640, the calculated vapor pressure may then be compared to the target vapor pressure specified. If the vapor pressure is at target or within a specified tolerance from target, operations may continue as currently operating in Block 650, with no need to adjust operating parameters. If the vapor pressure is outside the target range, then operations may be adjusted, in Block 660, to bring the processed fluids back to the target vapor pressure. Further, based on the adjustments to the operations in the Block 660, the previously mentioned Blocks (500-540) may then be repeated.

The computer system/control system may also be configured, as noted above, to operate the system using feedback or feedforward control. Input or measured feed compositions may be used to feed forward initial control parameters for the initial separators, for example. Final processed fluid vapor pressure calculations may be used, for example, in feedback control of initial separator, heater treater, or stabilizer operations. During typical operations, a one-unit adjustment is typically made, where the vapor pressure of the final product is adjusted by only lowering a pressure of the stabilizer, for example, removing additional light components from the liquids fed to the stabilizer from upstream operations. Using the systems herein, however, operations of the overall system may be considered when adjusting conditions to reach a vapor pressure target—a small change in the conditions in the initial separators may have a meaningful impact on vapor pressure of the fluids recovered from the stabilizer, and such may be more economical than trying to remove lighter components from the stabilizer. Control systems herein may account for such based on the calculations associated with each stage of operations.

As one skilled in the art recognizes, processing of fluids necessarily involves fluctuations around a set point, be it temperature, pressure, flow rate, or other process variables. Likewise, the end processed product liquids recovered from the system may vary in vapor pressure. However, such products are generally accumulated in a tank for temporary storage before being shipped to a downstream processing system or an end user. While operating parameters may be used to estimate a vapor pressure of a flow stream, such as that of the liquids from a stabilizer, it is further contemplated that systems herein may calculate a vapor pressure of the accumulated end processed liquids. In such a manner, fluctuations resulting in a portion of the accumulated liquid having a high or higher than target vapor pressure may be accounted for by adjusting conditions (Block 540 and 660) to produce fluids having a lower than target vapor pressure, such that the bulk accumulated processed fluids may have a vapor pressure at or near the targeted vapor pressure, suitable to meet regulatory or customer requirements.

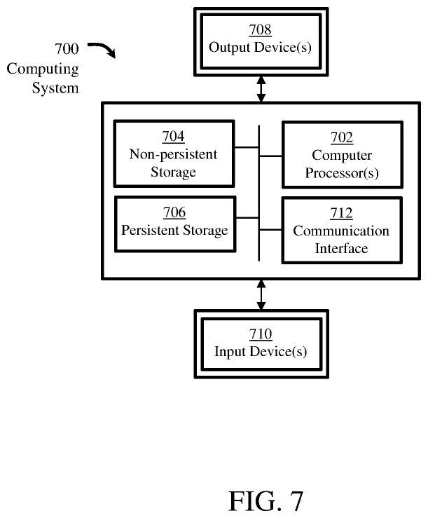

Implementations herein for operating the vapor pressure monitoring system 100 may be implemented on a computing system coupled to a controller in communication with the various components of the vapor pressure monitoring system 100. Any combination of mobile, desktop, server, router, switch, embedded device, or other types of hardware may be used with the vapor pressure monitoring system 100. For example, as shown in FIG. 7, the computing system 700 may include one or more computer processors 702, non-persistent storage 704 (e.g., volatile memory, such as random access memory (RAM), cache memory), persistent storage 706 (e.g., a hard disk, an optical drive such as a compact disk (CD) drive or digital versatile disk (DVD) drive, a flash memory, etc.), a communication interface 712 (e.g., Bluetooth interface, infrared interface, network interface, optical interface, etc.), and numerous other elements and functionalities. It is further envisioned that software instructions in a form of computer readable program code to perform embodiments of the disclosure may be stored, in whole or in part, temporarily or permanently, on a non-transitory computer readable medium such as a CD, DVD, storage device, a diskette, a tape, flash memory, physical memory, or any other computer readable storage medium. For example, the software instructions may correspond to computer readable program code that, when executed by a processor(s), is configured to perform one or more embodiments of the disclosure.

The computing system 700 may also include one or more input devices 710, such as a touchscreen, keyboard, mouse, microphone, touchpad, electronic pen, or any other type of input device. Additionally, the computing system 700 may include one or more output devices 708, such as a screen (e.g., a liquid crystal display (LCD), a plasma display, touchscreen, cathode ray tube (CRT) monitor, projector, or other display device), a printer, external storage, or any other output device. One or more of the output devices may be the same or different from the input device(s). The input and output device(s) may be locally or remotely connected to the computer processor(s) 702, non-persistent storage 704, and persistent storage 706. Many different types of computing systems exist, and the input and output device(s) may take other forms.

The computing system 700 of FIG. 7 may include functionality to present raw and/or processed data, such as results of comparisons and other processing. For example, presenting data may be accomplished through various presenting methods. Specifically, data may be presented through a user interface provided by a computing device. The user interface may include a GUI that displays information on a display device, such as a computer monitor or a touchscreen on a handheld computer device. The GUI may include various GUI widgets that organize what data is shown as well as how data is presented to a user. Furthermore, the GUI may present data directly to the user, e.g., data presented as actual data values through text, or rendered by the computing device into a visual representation of the data, such as through visualizing a data model. For example, a GUI may first obtain a notification from a software application requesting that a particular data object be presented within the GUI. Next, the GUI may determine a data object type associated with the data object, e.g., by obtaining data from a data attribute within the data object that identifies the data object type. Then, the GUI may determine any rules designated for displaying that data object type, e.g., rules specified by a software framework for a data object class or according to any local parameters defined by the GUI for presenting that data object type. Finally, the GUI may obtain data values from the data object and render a visual representation of the data values within a display device according to the designated rules for that data object type.

Data may also be presented through various audio methods. Data may be rendered into an audio format and presented as sound through one or more speakers operably connected to a computing device. Data may also be presented to a user through haptic methods. For example, haptic methods may include vibrations or other physical signals generated by the computing system. For example, data may be presented to a user using a vibration generated by a handheld computer device with a predefined duration and intensity of the vibration to communicate the data.

In addition to the benefits described above, the vapor pressure monitoring system 100 may further beneficially provide continuous measurements of the vapor pressure of the fluids. By providing continuous vapor pressure measurements, the vapor pressure monitoring system 100 may detect real-time changes to the fluid that would affect the final product. Further, the vapor pressure monitoring system 100 may allow for real-time adjusts when changes are detected to avoid costly NPT and mitigate the risk of the final product being rejected. In addition, the continuous monitoring of the vapor pressure allows for optimization so that the fluid may treated (e.g., heated) enough to meet vapor pressure requirements without unnecessary treatments. Furthermore, the vapor pressure monitoring system 100 does not require any maintenance other than maintenance on the plurality of sensors which reduces cost and personnel required on site.

While the method and apparatus have been described with respect to a limited number of embodiments, those skilled in the art, having benefit of this disclosure, will appreciate that other embodiments can be devised which do not depart from the scope as disclosed herein. Accordingly, the scope should be limited only by the attached claims.

What is claimed is:

1. A vapor pressure monitoring system comprising:
a plurality of sensors disposed on equipment at a processing facility during one or more stages of refining and/or processing fluids, wherein the plurality of sensors is configured to monitor one or more properties of the fluid; and
one or more transmitters configured to transmit the one or more properties from the plurality of sensors to a computer system,
wherein the computer system is configured to determine vapor pressure of the fluids based on the one or more fluid properties.

2. The vapor pressure monitoring system of claim 1, wherein the computer system is configured to display the determined vapor pressure for a user to access.

3. The vapor pressure monitoring system of claim 2, wherein the computer system is configured to send alerts to the user and/or automatically adjust, with a controller of the computer system, the equipment at the processing facility.

4. The vapor pressure monitoring system of claim 1, wherein the determined vapor pressure is a Reid vapor pressure or a true vapor pressure.

5. The vapor pressure monitoring system of claim 1, wherein the one or more fluid properties is a temperature, a pressure, a density, a flow rate, or a viscosity of the fluid.

6. The vapor pressure monitoring system of claim 1, wherein the equipment is one or more separators, one or more heater treaters, and one or more tanks.

7. The vapor pressure monitoring system of claim 1, wherein the fluids are gasoline, crude oil, or other petroleum products.

8. A method comprising:
monitoring one or more fluid properties of a fluid with a plurality of sensors at one or more stages in a processing facility;
transmitting the one or more fluid properties, via one or more transmitters, to a computer system; and
determining, with the computer system, vapor pressure of the fluid based on the one or more fluid properties.

9. The method of claim 8, further comprising controlling, with a controller coupled to the computer system, an operation of the one or more stages.

10. The method of claim 9, further comprising adjusting, with the controller, the operation of the one or more stages to change the determined vapor pressure.

11. The method of claim 8, further comprising displaying the determined vapor pressure on a display coupled to the computer system.

12. The method of claim 8, wherein the monitoring of the one or more fluid properties comprises measuring a temperature, a pressure, a density, a flow rate, or a viscosity of the fluid.

13. The method of claim 8, wherein the determining of the vapor pressure comprises cross-corelating the one or more fluid properties in a three-dimensional lookup table.

14. The method of claim 13, wherein the three-dimensional lookup table contains pre-determined or measured values for a vapor pressure based on different fluid property values.

15. The method of claim 8, further comprising continuously monitoring the one or more fluid properties of the fluid.

16. A non-transitory computer readable medium storing instructions on a memory coupled to a processor, the instructions comprising functionality for:
obtaining one or more fluid properties of a fluid at one or more stages in a processing facility;
wherein the processor is configured to:
determine vapor pressure of the fluid based on the one or more fluid properties; and
display the determined vapor pressure on a display coupled to the processor.

17. The non-transitory computer readable medium of claim 16, wherein the instructions further comprise functionality for:
comparing, with the processor, the determined vapor pressure to a required specification; and
sending alerts, with the processor, based on the determined vapor pressure meeting the required specification.

18. The non-transitory computer readable medium of claim 17, wherein, when the determined vapor pressure does not meet the required specification, the instructions further comprise functionality for:
adjusting, with the processor, an operation to change the one or more fluid properties until the determined vapor pressure meets the required specification.

19. The non-transitory computer readable medium of claim 16, wherein the instructions further comprise functionality for:
sending commands, with the processor, to equipment of the one or more stages to maintain or adjust the one or more fluid properties; and
re-determining, with the processor, vapor pressure of the fluid based on the maintained or adjusted one or more fluid properties.

20. The non-transitory computer readable medium of claim 16, wherein the instructions further comprise functionality for:
continuously determining, with the processor, the vapor pressure of the fluid in real-time.
